(19) United States
(12) Patent Application Publication
(10) Pub. No.: US 2025/0221726 A1
(43) Pub. Date: Jul. 10, 2025

(54) SUBCUTANEOUS ABRASION TREATMENT FOR STRETCH MARKS

(71) Applicant: The Children's Medical Center Corporation, Boston, MA (US)

(72) Inventor: Arin K. Greene, Wellesley, MA (US)

(73) Assignee: The Children's Medical Center Corporation, Boston, MA (US)

(21) Appl. No.: 18/703,100

(22) PCT Filed: Oct. 19, 2022
(86) PCT No.: PCT/US2022/047138

§ 371 Date: Apr. 19, 2024

Related U.S. Application Data
(60) Provisional application No. 63/257,881, filed on Oct. 20, 2021.

(51) Int. Cl. A61B 17/32; A61B 17/00

(52) U.S. Cl. CPC A61B 17/32; A61B 2017/00761; A61B 2017/320004

(57) ABSTRACT

Devices and methods for subcutaneously abrading an inner surface of a dermis of a subject are disclosed herein. A subcutaneous abrasion device may include a handle. an elongated shaft extending distally from the handle, and an abrasive surface disposed on a distal end portion of the elongated shaft. The abrasive surface may be configured to subcutaneously abrade an inner surface of a dermis of a subject without substantially cutting or puncturing the dermis of the subject.

### RELATED APPLICATION

This application claims the benefit under 35 U.S.C. § 119 (e) to U.S. Provisional Application No. 63/257,881 filed on Oct. 20, 2021 and entitled “SUBCUTANEOUS ABRASION TREATMENT FOR STRETCH MARKS,” the entire contents of which are incorporated herein by reference.

### FIELD

Disclosed embodiments relate to devices and methods for subcutaneously abrading a section of skin of a subject, for example to treat stretch marks.

### BACKGROUND

Stretch marks (striae distensae) are common skin scars that affect almost one-half of humans, including approximately 40% of males and approximately 70% of females. The appearance of the scars may cause a cosmetic concern, among others, particularly for patients who do not like the appearance of the scars.

### BRIEF SUMMARY

According to one aspect, a subcutaneous abrasion device includes a handle; an elongated shaft extending distally from the handle; and an abrasive surface disposed on a distal end portion of the elongated shaft, wherein the abrasive surface is configured to subcutaneously abrade an inner surface of a dermis of a subject without substantially cutting or puncturing the dermis of the subject.

According to another aspect, a method of treating a section of skin of a subject includes inserting an abrasion device into an opening in the skin of the subject; and subcutaneously abrading an inner surface of a dermis of the subject.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is a side view of a subcutaneous abrasion device according to one illustrative embodiment;

FIG. 1B is a side view of a subcutaneous abrasion device according to one illustrative embodiment;

FIG. 1C is a side view of a subcutaneous abrasion device configured to selectively transition between an undeployed and deployed configuration according to one illustrative embodiment;

FIG. 1D is a side view of a subcutaneous abrasion device configured to selectively transition between an undeployed and deployed configuration according to one illustrative embodiment;

FIG. 1E is a side view of a subcutaneous abrasion device according to one illustrative embodiment;

FIG. 1F is a side view of a subcutaneous abrasion device according to one illustrative embodiment;

FIG. 1G is a side view of a subcutaneous abrasion device according to one illustrative embodiment;

FIG. 2 is a side view of a subcutaneous abrasion device in use on a subject according to one illustrative embodiment; and

FIG. 3 is a flowchart illustrating a method of using a subcutaneous abrasion device according to one illustrative embodiment.

### DETAILED DESCRIPTION

Generally speaking, when the skin of a subject stretches, a scar known as a stretch mark may appear in the skin. In particular, stretch marks may form when the collagen and/or elastin that supports the skin is damaged or ruptures. Stretch marks may form under any appropriate circumstances, including during growth spurts, pregnancy, rapid weight loss or gain, rapid muscle growth, and/or other appropriate circumstances. Stretch marks may cause a cosmetic concern, among others, particularly for patients who do not like the appearance of the stretch marks.

Conventional therapies for treating stretch marks include applying a treatment or other procedure to the top (e.g., the exterior outwardly facing) surface of the skin. These treatments may include the following: topical medicine (e.g., Mederma, retinoids, silicone), chemical peeling, microdermabrasion, lasers (e.g., non-ablative lasers (e.g., pulsed dye, 1450 diode, 1550 erbium) and/or ablative lasers (e.g., CO₂, erbium/yag)), radiofrequency, and/or microneedling. Such treatments and procedures may have variable efficacy, morbidity, and/or a need for multiple treatments. Moreover, such “top down” approaches seek to reach the dermis through the epidermis and/or induce damage in a limited upper portion of the dermis (e.g., the papillary layer of the dermis or a small upper portion of the reticular layer of the dermis), that may be repaired with the formation of new collagen in the dermis to treat the presence of stretch marks. However, it may be challenging for such “top down” approaches to effectively penetrate through the skin to the lower portions of the dermis to induce the desired regeneration effects (e.g., collagen growth), which may limit the effectiveness of such “top down” approaches.

In view of the above, the inventor has recognized the advantages of a subcutaneous approach to promoting repair of the dermis, for example to treat stretch marks. In particular, according to some aspects of the present disclosure, a method of subcutaneously treating the skin of a subject (e.g., to treat stretch marks) is disclosed. In some embodiments, a clinician may insert an abrasion device into an opening in the skin of the subject, thus allowing the abrasion device to reach an inner surface of a dermis of a subject. Accordingly, the clinician may then subcutaneously abrade the inner surface of the dermis of the subject (e.g., the deepest reticular layer at its junction with the adipose tissue below it), inducing the skin of the subject to heal (e.g., by repairing the skin with new collagen as described herein). As elaborated on below, such a method is able to directly affect the lower portions of the dermis as compared to prior systems and methods which are limited to treating an upper portion of the epidermis or dermis (e.g., the papillary layer of the dermis or a small upper portion of the reticular layer of the dermis).

According to another aspect, a subcutaneous abrasion device is disclosed. The subcutaneous abrasion device may include a handle, an elongated shaft, and an abrasive surface. The elongated shaft may extend distally from the handle, and in turn, the abrasive surface may be disposed on a distal end portion of the elongated shaft. In some embodiments, the abrasive surface may be configured to subcutaneously abrade an inner surface of a dermis of a subject without substantially cutting or puncturing the dermis of the subject.

Unlike conventional treatments that may be limited to treating an upper portion of the dermis of a subject (e.g., the papillary layer or a small upper portion of the reticular layer of the dermis), the embodiments disclosed herein may provide for direct access to lower portions of the dermis, which include the reticular layer of the dermis. Thus, the embodiments disclosed herein may provide a clinician with the ability to more easily and directly induce stresses in the underside of the dermis. Additionally, the Inventor has found that such a treatment method is capable of inducing large scale restructuring of the dermis to more effectively treat stretch marks as compared to other “top down” methods.

As will be appreciated by one of skill in the art, in addition to directly inducing stresses on the underside of the dermis, subcutaneously abrading the underside of the dermis directly may allow a clinician to affect a larger portion of the dermis in a shorter time period. For example, a chair time (e.g., a time it takes for a procedure to be performed) may be reduced relative to conventional treatments, such as those described herein. In some instances, the chair time may be reduced from approximately 45 minutes to approximately 10 minutes, though any appropriate treatment time may be used depending on the size and extent of a procedure as the disclosure is not so limited.

As described herein, and without wishing to be bound by theory, subcutaneously abrading a dermis of a subject may promote increased collagen generation in the subject in the abraded areas. Increased collagen generation in the abraded areas may drastically reduce the visibility of stretch marks, particularly in comparison to conventional treatments.

In some embodiments, the subcutaneous abrasion may be performed in a minimally invasive manner. For example, a clinician may make a small incision in a subject to create an opening in the subject's skin. The clinician may then insert an abrasion device into the opening to scrape and/or rub an abrasive surface of the abrasion device against a target inner surface of the dermis of the subject to directly induce stresses on the underside of the dermis. The abrasion device may then be removed via the same opening. In some embodiments, the abrasion device may be deployable within the subject so that an abrasive surface is deployed from a smaller undeployed configuration that is smaller than the opening to a deployed configuration that is larger than the undeployed configuration and the opening. In some instances, the opening may be created at or near the navel, or other appropriate portion, of the subject's skin to blend the incision with preexisting lines within a body of the subject. Of course, it should be understood that while a deployable abrasive surface is discussed above fixed abrasive surfaces are also contemplated as elaborated on below.

As will be appreciated by one of skill in the art, while a clinician may seek to induce stresses and/or microtears in the dermis (e.g., as described herein), it may be desirable for the dermis to remain substantially intact during a treatment (e.g., substantially free from punctures, cuts, tears, etc.). Accordingly, a subcutaneous abrasion device according to the present disclosure may include an abrasive surface in any suitable size and/or shape that is configured to abrade an inner surface of the dermis of a subject while substantially avoiding punctures, cuts, tears, and other large scale damage to the skin of a subject. In some embodiments, the abrasive portion may have any appropriate shape including, but not limited to rectangular, circular, cylindrical, spherical, semicircular and/or any other suitable shape. Alternatively or in addition, the abrasive portion may be wider than an elongated shaft of the subcutaneous abrasion device, though this need not be the case. However, embodiments with abrasive portions less wide than the elongated shaft it is associated with, or equally as wide as the elongated shaft, are also contemplated, as the disclosure is not so limited.

Moreover, the abrasive surface may include any suitable feature or features configured to abrade an inner surface of a subject's dermis. For example, in some embodiments, the abrasive surface may include one or more: rasps; barbs; protrusions; angled surfaces; fins; structures including edges that extend radially outwards from an associated portion of an elongated shaft, and/or other appropriate structures that are configured to introduce stresses onto the inner sections of the dermis while avoiding puncturing or tearing of the dermis during normal operation.

In some embodiments, the abrasive surface may extend at least partially in a distal direction relative to an elongated shaft of a device, but the abrasive surface may by angularly offset relative to the elongated shaft of the subcutaneous abrasion device by an offset angle. In some embodiments, the offset angle may be greater than or equal to 30 degrees, 40 degrees, 50 degrees, and/or any other appropriate dimension. In some embodiments, the offset angle may be less than or equal to 90 degrees, 80 degrees, 70 degrees, and/or any other appropriate dimension. Combinations of the foregoing are contemplated including, for example, an offset angle that is between or equal to 30 degrees and 90 degrees. Of course, the offset angle may take on any suitable value, depending on the application, as the disclosure is not limited in this regard.

In some embodiments, the offset angle may be adjustable, while in other embodiments the offset angle may be fixed. In embodiments where the offset angle is adjustable, the offset angle may be actively adjustable (e.g., affirmatively by a clinician) and/or passively adjustable (e.g., based on the contours of an inner surface of a dermis of a subject).

In some embodiments, a subcutaneous abrasion device according to the present disclosure may include an abrasive surface with one or more automated or movable structures. For example, in some embodiments, the abrasive surface may include a spinning member. The spinning member may be driven electrically (e.g., by an electric motor) or passively (e.g., based on the contours of the skin of a subject). Of course, any movable structure of the abrasive surface may be arranged in any suitable manner as structures other than spinning members are also contemplated. Such an automated or movable member may serve to reduce the repetition of strokes during treatment (e.g., by the clinician) and/or increase the consistency of results between different subjects.

As will be appreciated by one of skill in the art, in some embodiments, it may be desirable to shield a patient from the abrasive surface of a subcutaneous abrasion device until the abrasive surface is positioned proximate to a target inner surface of a dermis of a subject. Accordingly, a clinician may insert the subcutaneous abrasion device into an opening of the subject with the abrasive surface facing away from the inner surface of the dermis. Then, once the abrasive surface is appropriately positioned, the clinician may rotate the subcutaneous abrasive device such that the abrasive surface contacts the target inner surface of the dermis of the subject. In such an embodiment, the device may have a reduced dimension in one direction to facilitate insertion in a first orientation and a larger dimension in a second direction to facilitate treating a desired target area of a subject's skin.

In some embodiments, the abrasive surface of the subcutaneous abrasion device may be deployable. For example, in an undeployed configuration, the abrasive surface may have a first smaller transverse dimension (i.e., width or diameter). Accordingly, in such a state, a clinician may easily and minimally invasively insert the subcutaneous abrasion device into a subject (e.g., as described herein). Once the subcutaneous abrasion device is inserted into a subject, the abrasive surface may then be deployed (e.g., either actively or passively) to a deployed configuration such that the abrasive surface has a larger transverse dimension as compared to the undeployed configuration. In some instances the larger transverse dimension may be wider than the elongated shaft of a device.

In some embodiments, a length of an elongated shaft of a subcutaneous abrasion device may be adjustable. For example, the elongated shaft may be configured to telescopically transition between different lengths (e.g., a first retracted length and a second fully extended length), depending on the application. Of course, this need not be the case as embodiments with fixed elongated shafts are also contemplated.

Turning to the figures, specific non-limiting embodiments are described in further detail. It should be understood that the various systems, components, features, and methods described relative to these embodiments may be used either individually and/or in any desired combination as the disclosure is not limited to only the specific embodiments described herein.

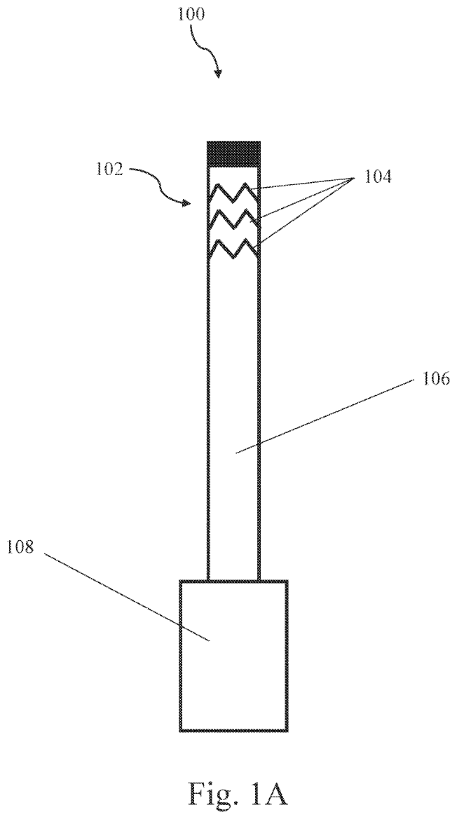

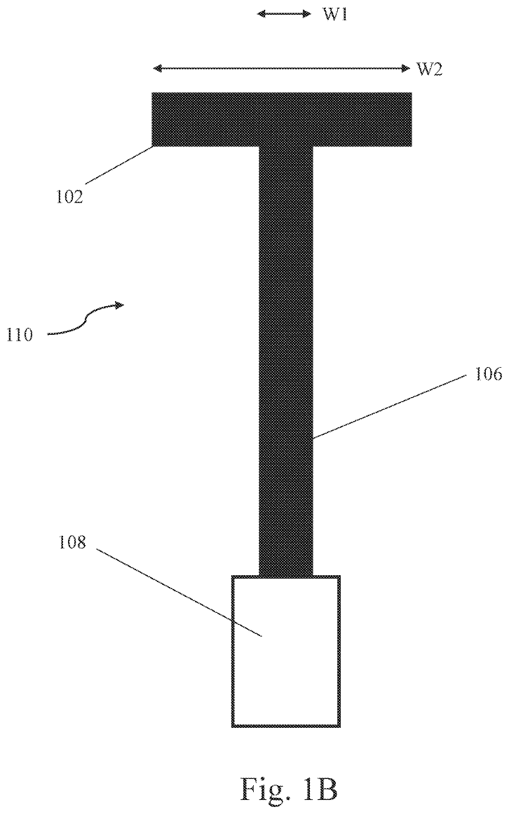

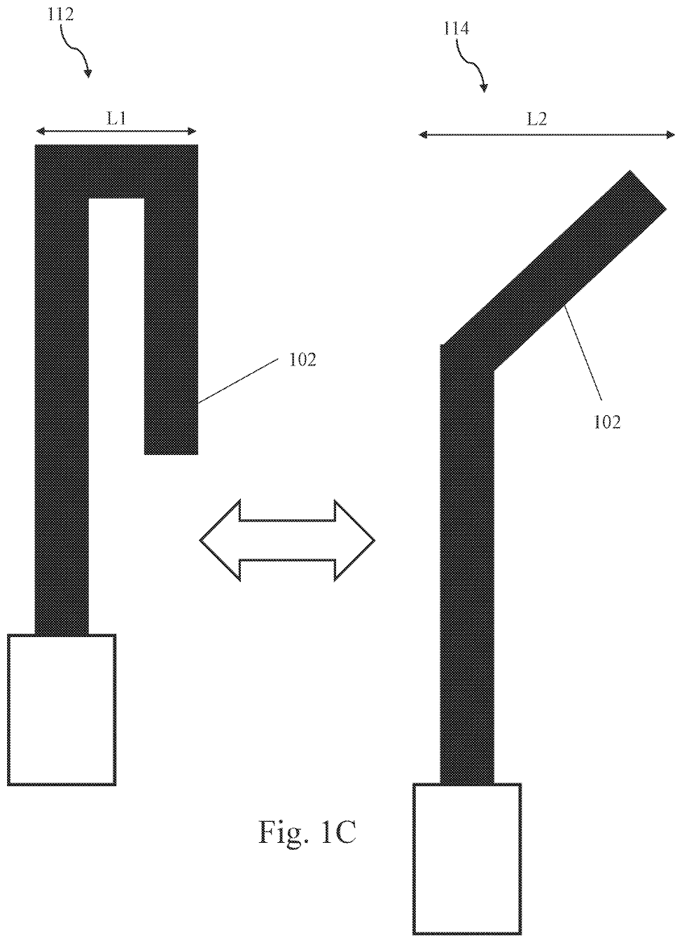

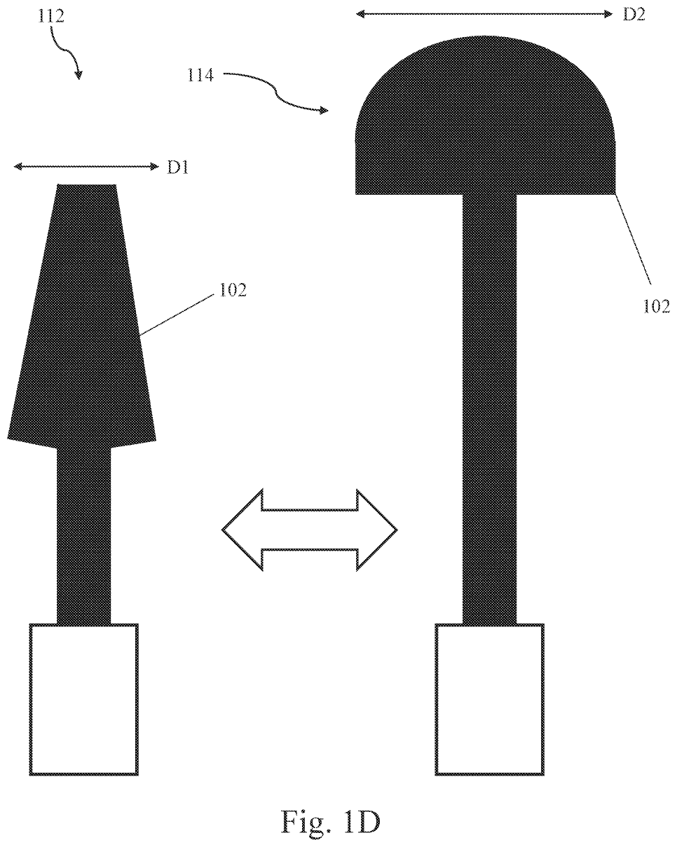

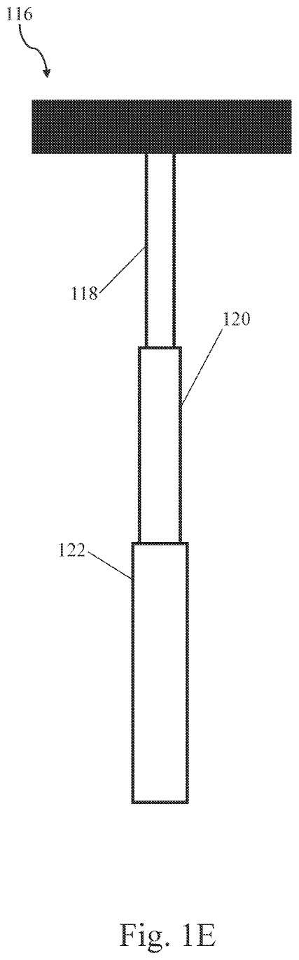

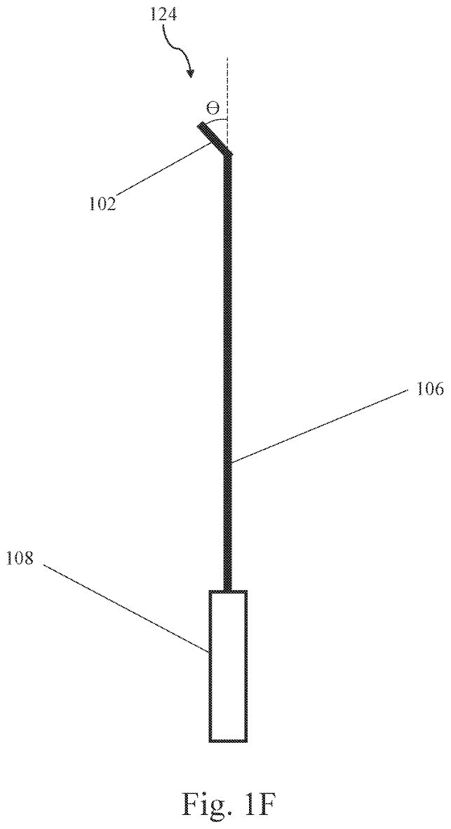

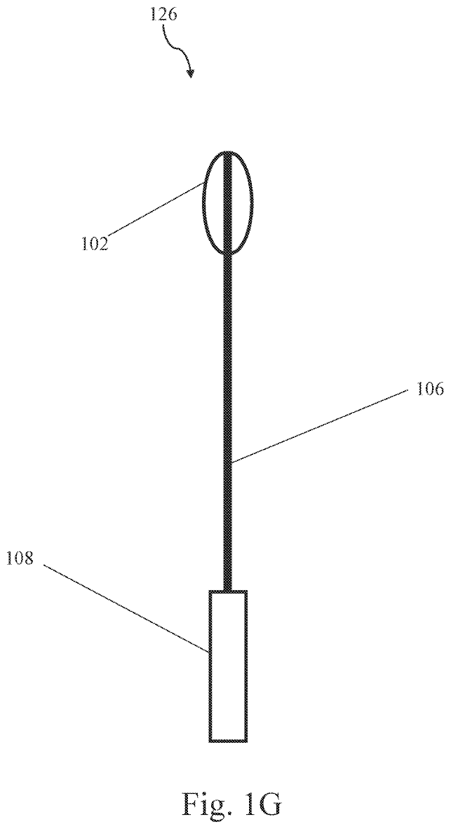

FIG. 1A shows a subcutaneous abrasion device 100 according to one illustrative embodiment. The subcutaneous abrasion device 100 includes a handle 108, an elongated shaft 106, and an abrasive surface 102. The elongated shaft 106 extends distally away from the handle 108. The handle 108 includes an abrasive surface 102 disposed on a distal end of the elongated shaft 106. In the illustrated embodiment, the abrasive surface includes a plurality of rasps 104 configured to non-destructively (e.g., as described herein) abrade an inner section of a dermis of a subject.

FIG. 1B shown a subcutaneous abrasion device 110 configured to reduce the chair time of a subject during a procedure. Similar to the above, the abrasive surface 102 is disposed on a distal portion of an elongated shaft 106 attached to a handle 108. However, the abrasive surface is configured to be wider than the elongated shaft 106. In particular, the abrasive surface 102 has a width W2 that is greater than a width W1 of the elongated shaft 106. In the depicted embodiment, the abrasive surface corresponds to an elongated paddle disposed on a distal portion of the elongated shaft and extending in a direction that is at least partially transverse to a longitudinal axis of the elongated shaft. The wider abrasive surface 102 may allow a clinician to abrade a greater area of an inner surface of a dermis of a subject for a given dexterous motion (e.g., via the handle as described herein). Thus, a clinician may be able to abrade a target section of an inner surface of a dermis of a subject more quickly and a chair time for the subject may be reduced.

In some embodiments, it may be desirable for treatment associated with a subcutaneous abrasion device to be administered in a minimally invasively manner. Thus, a subcutaneous abrasion device may be deployable, as shown in the embodiment of FIGS. 1C-1D. In particular, it may be desirable for the subcutaneous abrasion device to deploy from an undeployed configuration with a smaller width to a deployed configuration with a larger width (e.g., to reduce chair time as described herein). For example, as shown in FIG. 1C, in some embodiments, an abrasive surface 102 of a subcutaneous abrasion device may be capable of deploying from a first folded configuration to a deployed configuration with the abrasive surface extending at least partially outward from the elongated shaft. However, other deployable shapes may also be used including a deployable T-shape or paddle disposed on a distal portion of the elongated shaft. In the depicted embodiment, the subcutaneous abrasion device may be initially set in an undeployed configuration 112. In the undeployed configuration 112, the abrasive surface 102 of the subcutaneous abrasion device has a first width L1. The subcutaneous abrasion device may be inserted into a subject in the undeployed configuration, for example, to allow for a smaller incision to be made in the subject (e.g., as compared to a deployed subcutaneous abrasion device). Once inserted, the abrasive surface 102 of the subcutaneous abrasion device may be deployed into a deployed configuration 114. In the deployed configuration 114, the abrasive surface 102 of the subcutaneous abrasion device has a second width L2 greater than the first width L1.

Relatedly, as shown in the embodiment of FIG. 1D, in some embodiments, an abrasive surface 102 of a subcutaneous abrasion device may be capable of deploying into an umbrella shape. As with the embodiment of FIG. 1C, in the depicted embodiment, the subcutaneous abrasion device may be initially set in an undeployed configuration 112. In the undeployed configuration 112, the abrasive surface 102 of the subcutaneous abrasion device has a first width D1. The subcutaneous abrasion device may be inserted into a subject in the undeployed configuration, for example, to allow for a smaller incision to be made in the subject (e.g., as compared to a deployed subcutaneous abrasion device). Once inserted, the abrasive surface 102 of the subcutaneous abrasion device may be deployed into a deployed configuration 114. In the deployed configuration 114, the abrasive surface 102 of the subcutaneous abrasion device has a second width D2 greater than the first width D1.

In some embodiments, it may be desirable for the elongated shaft of a subcutaneous abrasion device to have an adjustable length. For example, as depicted in FIG. 1E, in some embodiments, a subcutaneous abrasion device 116 includes an elongated shaft having multiple telescoping shaft portions 118, 120, 122. As will be appreciated, the first telescoping shaft portion 118 may be configured to fit within the second telescoping shaft portion 120. In turn, the second telescoping shaft portion 120 and/or the first telescoping shaft portion 118 may be configured to fit within the third telescoping shaft portion 122 in a collapsed configuration, though other telescoping configurations may also be used. Thus, a clinician may adjust a length of the subcutaneous abrasion device 116, for example to adapt the device to different applications.

In some embodiments, it may be desirable for a subcutaneous abrasion device to include an angled abrasive surface, for example, to dexterously manipulate the subcutaneous abrasion device in otherwise difficult to reach locations within a subject. One such embodiment is depicted in FIG. 1F. FIG. 1F shows a subcutaneous abrasion device 124 having an abrasive surface 102 angularly offset from an elongated shaft 106 by an offset angle Θ. The offset angle Θ may be adjustable (either actively or passively as described herein). In some embodiments, the offset angle may be adjusted based on inputs from a clinician on a handle 108.

FIG. 1G depicts yet another embodiment of a subcutaneous abrasion device 126 having an abrasive surface 102 disposed on a distal portion of the elongated shaft 106 and a handle 108 that the elongated shaft extends distally from. In the depicted embodiment, the abrasive surface corresponds to one or more, and in some instances a plurality of, structures extending radially outwards from an associated portion of an elongated shaft which may have a closed outer surface in at least this portion of the device. The structures may include one or more edges that are configured to abrade the inner surface of a dermis of the subject. In the specific embodiment shown in the figure, the structures correspond to curved strips that extend between a first proximal location and a second distal location along a length of the elongated shaft. Between these locations, the strips are disposed radially outwards from the elongated shaft such that one or more edges of the strips may be engaged with adjacent tissue to abrade the tissue as the device is moved. Of course, while a curved shape is shown other shapes including polygonal shapes are also contemplated as the disclosure is not limited to a shape of the depicted structures extending radially outward from the elongated shaft. Additionally, while strips are depicted in the figure with a gap disposed between portions of the strips and shaft are illustrated, embodiments in which solid projections with edges located at a position disposed radially outward from a surface of the elongated shaft are also contemplated.

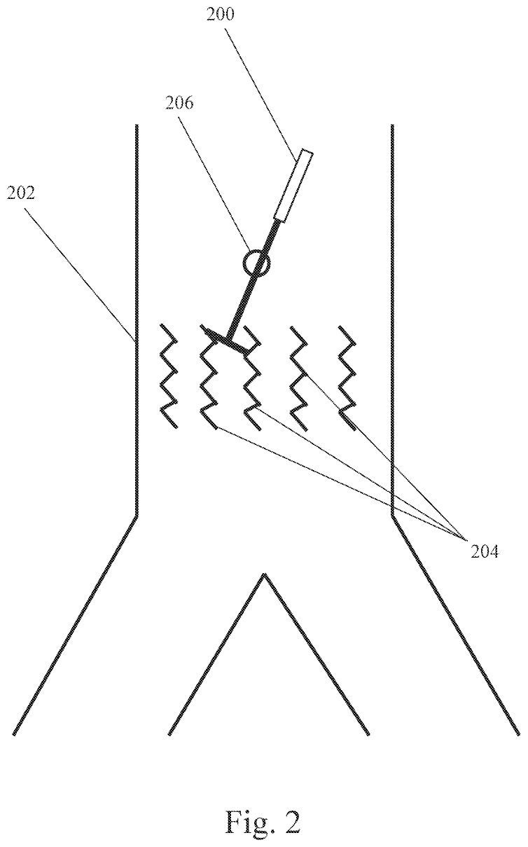

FIG. 2 shows a subcutaneous abrasion device 200 being used to treat a subject 202. To treat the subject 202, the clinician may create an opening 206 (e.g., an incision) in the subject 202. The clinician may then insert the subcutaneous abrasion device 200 into the subject 202 via the opening 206 and guide an abrasive surface of the subcutaneous abrasion device 200 towards a target section of an inner surface of a dermis of the subject 202. In the depicted embodiment, the one or more target sections correspond to stretch marks 204, which may be treated by abrading the inner surface of the dermis with the depicted subcutaneous abrasion device. Once the target sections are treated, the clinician may remove the subcutaneous abrasion device 200 from the subject 202 via the opening 206 and seal the opening 206 (e.g., using stiches, adhesives, bandages, etc.).

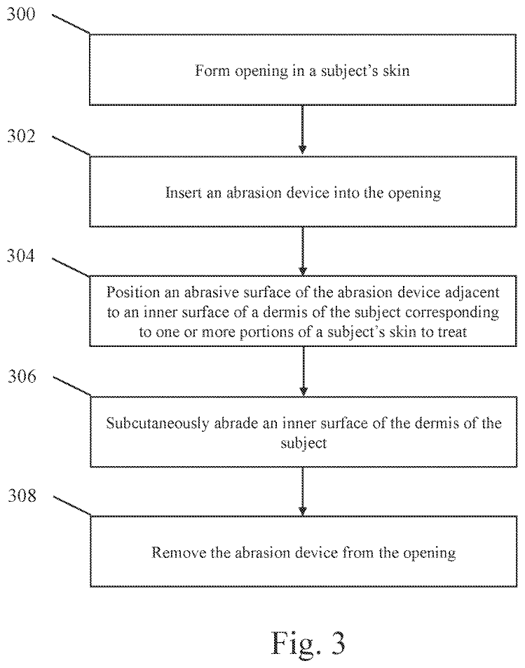

FIG. 3 is a flowchart showing a method of abrading an inner surface of a dermis of a subject. In particular, at step 300, a clinician may first form an opening in a subject's skin prior to inserting a subcutaneous abrasion device (e.g., according to the embodiments described herein) into an opening in the patient at 302. Subsequently at step 304, the clinician may position an abrasive surface of the subcutaneous abrasion device adjacent to an inner surface of the dermis of the subject corresponding to one or more portions of the subject's skin to treat. In some embodiments, this may optionally include a step of deploying the abrasive surface from a first undeployed configuration to a deployed configuration. In either case, at 306, the clinician may use the abrasive surface to abrade an inner surface of the dermis of the subject prior to removing the subcutaneous abrasion device from the opening of the subject at 308. In some embodiments, the method may further include removing the subcutaneous abrasion device from the subject, sealing the opening, and/or rotating the subcutaneous abrasion device, as described herein.

### Example: Subcutaneous Skin Abrasion During Liposuction Procedure

During a liposuction procedure performed for reconstructive purposes associated with a subject's condition, the Inventor used a liposuction cannula including aggressive surface features in a manner that is not typically done during liposuction procedures. Accordingly, the Inventor recognized that the cannula abraded an inner surface of the dermis of a subject. After the surgical procedure, it was recognized that the sections of the subject's skin that were subjected to this unintended treatment (i.e., abrasion of an inner surface of the dermis) showed a drastic reduction in stretch marks as compared to untreated sections of skin where the stretch marks remained. Since this initial observation, a reduction in the presence of stretch marks in response to abrading an inner surface of a subject's dermis has been experimentally observed in (1) different subjects, (2) different diagnoses (e.g., lipedema and gynecomastia), (3) different genders (e.g., biologically female and male subjects), (4) different anatomical locations (e.g. the legs and chest), and (5) multiple areas within the same subject (e.g., left and right legs as well as left and right sides of the chest). The observed results were initially unexpected as typical treatments for stretch marks are conducted in a top down fashion through the top layers of the skin while avoiding damage to the inner most layers of the dermis. The fact that these benefits have now been seen in multiple subjects, locations, and genders now confirms that the currently disclosed methods for subcutaneously abrading an inner surface of a dermis may provide effective treatment of stretch marks in a subject.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

While the present teachings have been described in conjunction with various embodiments and examples, it is not intended that the present teachings be limited to such embodiments or examples. On the contrary, the present teachings encompass various alternatives, modifications, and equivalents, as will be appreciated by those of skill in the art. Accordingly, the foregoing description and drawings are by way of example only.

1. A subcutaneous abrasion device comprising:
a handle;
an elongated shaft extending distally from the handle; and
an abrasive surface disposed on a distal end portion of the elongated shaft, wherein the abrasive surface is configured to subcutaneously abrade an inner surface of a dermis of a subject without substantially cutting or puncturing the dermis of the subject.

2. The subcutaneous abrasion device of claim 1, wherein the inner surface of the dermis includes a portion of a reticular layer of the dermis of the subject.

3. The subcutaneous abrasion device of claim 1, wherein the abrasive surface includes one or more rasps.

4. The subcutaneous abrasion device of claim 1, wherein the abrasive surface includes a head portion that is wider than the elongated shaft.

5. The subcutaneous abrasion device of claim 1, wherein the abrasive surface includes a deployable head that deploys from a first undeployed configuration to a second deployed configuration.

6. The subcutaneous abrasion device of claim 5, wherein the deployable head is selectively deployable within a subject.

7. The subcutaneous abrasion device of claim 1, wherein the elongated shaft is configured to have an adjustable length.

8. The subcutaneous abrasion device of claim 1, wherein the elongated shaft is a telescoping elongated shaft.

9. The subcutaneous abrasion device of claim 1, wherein at least the abrasive portion is configured to be inserted into an opening in the subject.

10. A method of treating a section of skin of a subject comprising:
inserting an abrasion device into an opening in the skin of the subject; and
subcutaneously abrading an inner surface of a dermis of the subject.

11. The method of claim 10, further including removing the abrasion device from the opening.

12. The method of claim 10, wherein inserting the abrasion device into the opening includes inserting the abrasion device with an abrasive surface facing away from the inner surface of the dermis.

13. The method of claim 12, further including rotating the abrasive surface towards the inner surface of the dermis after inserting the abrasion device with the abrasive surface facing away from the inner surface of the dermis and before subcutaneously abrading the inner surface of the dermis.

14. The method of claim 10, further including deploying an abrasive surface of the abrasion device from a first undeployed configuration to a second deployed configuration after inserting the abrasion device into the opening.

15. The method of claim 10, further including extending a length of an elongated shaft of the abrasion device.
